(19) United States
(12) Patent Application Publication
(10) Pub. No.: US 2007/0233206 A1
(43) Pub. Date: Oct. 4, 2007

(54) Method and arrangement for monitoring a medical appliance

(75) Inventors: Marcel Frikart, Bern (CH); Markus Jungen, Bolligen (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(73) Assignee: Disetronic Licensing AG

(21) Appl. No.: 11/729,065

(22) Filed: Mar. 28, 2007

(30) Foreign Application Priority Data
Mar. 29, 2006 (EP) 06006 502.6

(51) Int. Cl. A61N 1/00

(52) U.S. Cl. 607/60; 128/903

(57) ABSTRACT

A medical device adapted to transmit messages via a communication network to a communication device, forwarding the same or another message via the same or another communication network to a further communication device, and a method including producing a message in the medical device, transmitting the message to the communication device and forwarding the same or another message to another communication device, whereby the medical device may be remotely monitored.

### CROSS REFERENCE TO RELATED APPLICATION(S)

This application claims priority to European Patent Application No. 06006502.6, filed Mar. 29, 2006, the content of which is incorporated in its entirety by reference herein.

### BACKGROUND

The present invention relates to devices for delivering, infusing, injecting, administering or dispensing substances, and to methods of using and making such devices. It further relates to devices for measuring, testing or assessing substances, and to methods of using and making such devices. More particularly, it relates to a method for monitoring a medical appliance, such as an insulin pump and/or a blood sugar meter, and to arrangements or systems of components for monitoring insulin pumps and/or blood sugar meters.

Diabetics regularly need to measure their blood sugar level. When treating diabetes with an insulin pump, it is also necessary to control the amount of insulin which is released. For the purpose of checking the values, systems which can be operated by the diabetic are available which combine values from a meter and the infusion pump in order to give an overview and to allow the values to be interpreted. However, the regular measurement, pump control and interpretation of such measured values or messages may be an activity which can be carried out only with effort, particularly for children and older patients. This is where the present invention aims to bring about an improvement.

### SUMMARY

In one embodiment, the present invention provides improved regular measurement, appliance control and interpretation of measured values or messages.

In one embodiment, the present invention comprises a medical device adapted to transmit messages via a communication network to a communication device, the communication device adapted to send the same or another message via the same or another communication network to a further communication device, and a method including producing a message in the medical device, transmitting the message to the communication device and sending the same or another message to the further communication device, whereby the medical device may be remotely monitored.

In one embodiment, the present invention comprises a medical device adapted to transmit messages via a communication network to a communication device, which results in the communication device sending the same or another message via the same or another communication network to a further communication device, and a method including producing a message in the medical device, transmitting the message to the communication device and sending the same or another message to the further communication device, whereby the medical device may be remotely monitored.

In one embodiment, the present invention comprises an insulin pump designed or adapted to transmit messages via a short range radio network to a Smart phone, which results in the latter being caused or used for automatically sending a communication via a telephone network to a further telephone, thereby providing an arrangement and a method allowing the insulin pump to be remotely monitored automatically, which is advantageous for diabetic patients, including diabetic pediatric patients.

In one embodiment, the present invention provides a method comprising monitoring a medical appliance, e.g., an insulin pump, a blood sugar meter and/or a continuously measuring sensor, comprising the following steps:
producing a message in or associated with the medical appliance,
transmitting the message wirelessly via a short range radio link to a communication appliance which, for its part, is designed for wireless connection setup to a telephone or radio paging network,
forwarding the message via the telephone or radio paging network to a further wireless communication appliance connected to the same or a suitable telephone or radio paging network.

In one embodiment, the method of the present invention allows remote monitoring of an insulin pump and/or a blood sugar meter and/or a continuously measuring blood sugar sensor, e.g. by the parents of the pump wearer, by professional care-givers or by other suitable monitors (e.g., human, computers, electromechanical devices, etc.). The wireless, fully automated system, in which the transmission to the further communication appliance is initiated by the insulin pump and/or the blood sugar meter and/or the sensor, allows matters to be managed without any handling by or involvement of the pump wearer and/or user of the blood sugar meter. For patients who carry a mobile telephone, particularly a Smartphone, or a pager with them anyway, no expense is incurred for an additional appliance.

In some embodiments of the present invention, an insulin pump can firstly send a message to a mobile telephone or pager wirelessly and/or, in some embodiments, this can be done by a blood sugar meter. In one variant, the message from the blood sugar meter is also sent to the telephone or the pager wirelessly via the insulin pump.

### BRIEF DESCRIPTION OF THE DRAWINGS

FIG. 1 shows an example of a message transmission from an insulin pump; and

FIG. 2 shows an example of the message transmission from a blood sugar meter.

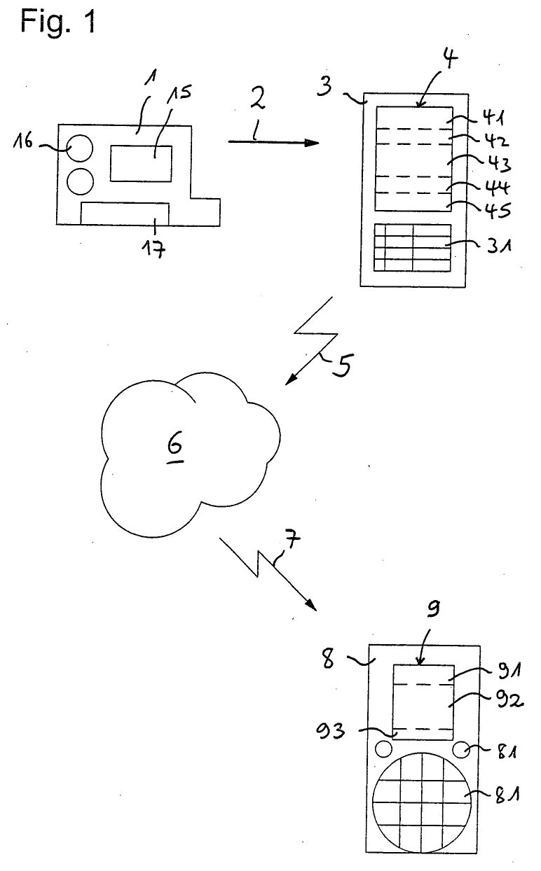

FIG. 1 shows a first exemplary embodiment of the present invention in which an insulin pump 1 is involved. The insulin pump has a display 15, operator control means 16 and a holder for the insulin ampoule, as is known. Such insulin pumps are known and do not require further description here with regard to their operation for releasing insulin to a patient. The insulin pump is thus in a form such that it can send the event messages and/or alarm messages occurring in known insulin pumps via a short-range radio link, for example a radio link based on the Bluetooth standard. This is shown in the figure by the arrow 2. The first communication appliance 3 is designed to receive these messages via the short range radio link. The insulin pump 1 can therefore set up a connection to the communication appliance 3 and send it the infusion pump messages, in some preferred embodiments via a Bluetooth radio link. In some preferred embodiments, the communication appliance 3 is a mobile telephone and, more particularly, may be what is known as a Smartphone, which executes an appropriate application for receiving and displaying the message from the insulin pump. Such Smartphones are commercially available and are known to have a keypad 31 and a display 4. It is likewise known that the mobile telephone is provided with a Bluetooth functionality or Bluetooth interface. The application accepts the message from the insulin pump and automatically generates a message which can be sent via the mobile telephone network. Besides the area 41, which shows the symbol bar for the operating system and for the application, the display 4 then shows, by way of example, the area 42, which indicates the status of the short range radio link, and the area 43, which indicates the transmission of an SMS message, for example, and also the areas 44 and 45, which indicate messages and values from the insulin pump. At least one of the messages from the insulin pump can be sent to the further appliance 8. This can be done such that each message sent to the appliance 3 by the insulin pump 1 is sent automatically, or the application on the appliance 3 may be in the form such it makes a decision regarding whether the message is a message which is relevant to the remote monitoring of the insulin pump 1 and which needs to be forwarded or whether it is another message which is merely displayed on the display 4 of the appliance 3 and at most stored therein. To this end, the application on the appliance 3 can contain a table of messages from an insulin pump and/or from a blood sugar meter which classifies these messages as messages to be forwarded and as messages which are not to be forwarded and uses such a table as a basis for making the decision regarding whether the appliance 3 forwards the message to the further appliance 8.

If it is a message which is intended to be forwarded to third parties or a remote location (e.g., a computer display or memory, database, etc.) for the purpose of remote monitoring, the appliance 3 or the application software running on it generates a message which is transmitted via the GSM network or the UMTS network 6, in some preferred embodiments as a short message based on the SMS protocol, the MMS protocol or other suitable protocol. The figure shows that with the transmission path 5 to the network 6. This message may contain the relevant pump alarm in encoded form or in plain text form and/or the indication of the released bolus with the relevant value.

The mobile telephone network 6 or, in the case of pagers, the radio paging network based on the POCSAG format, is used to send the message to the further communication appliance 8, which is shown in the figure by means of the arrow 7. This appliance 8 is a mobile telephone without Smartphone characteristics, a Smartphone or a pager with a keypad 81, for example. The display 9 in the areas 91, 92, 93 of the appliance 8 then displays the SMS or MMS message containing the information from the insulin pump and, in some embodiments, additional interpretations and recommendations for third parties in the area 92, for example. The third party, who receives the message from the insulin pump via the appliance 8, can then react accordingly if required. Suitable further networks are the transmission structures known as personal network, local network or wide area network.

In another preferred embodiment, the sequence is the same as in FIG. 1, but the medical appliance 1 in this case is a blood sugar meter or a continuously measuring blood sugar sensor which can likewise forward alarm messages, blood sugar measured values or other data to third parties or to the communication appliance 8 via the communication appliance 3. In this case, the execution may be substantially or generally the same as that described on the basis of the message from the infusion pump.

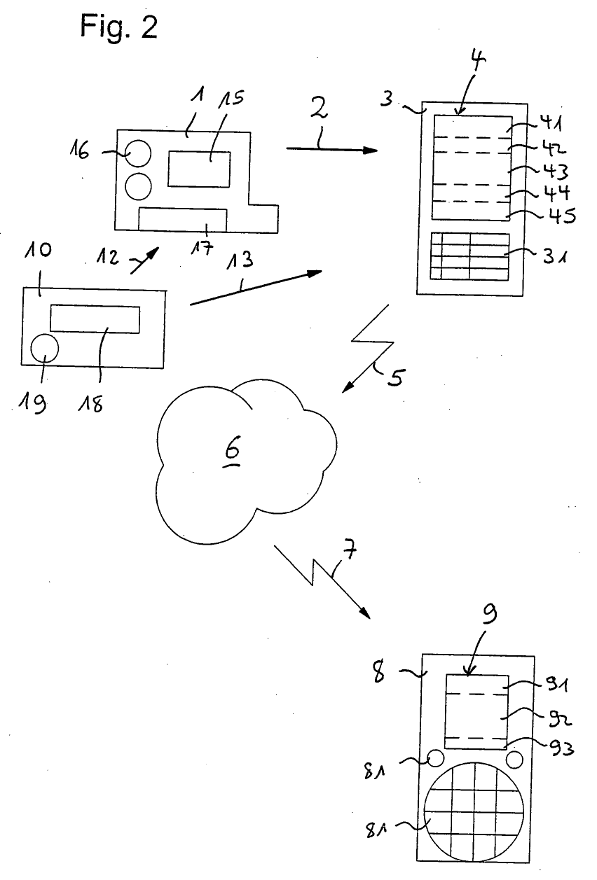

FIG. 2 shows another embodiment of the present invention, in which, in addition to the infusion pump 1, a blood sugar meter 10, or a continuous blood sugar sensor, with a display 18 and operator control elements 19 is shown. In this embodiment, the form is such that the blood sugar meter 10 communicates with the insulin pump via a short range radio link, a Bluetooth radio link or other suitable communication link, which is shown by means of the arrow 12. This may involve one-way communication from the blood sugar meter to the insulin pump 1 or two-way communication between the appliances. In this context and in some preferred embodiments, the appliances also interchange identification information such that the insulin pump 1 and the blood sugar meter 10 are paired and do not react to the information from other appliances. A blood sugar measured value or an alarm message from the blood sugar meter 10 is therefore transmitted to the insulin pump 1. There, an evaluation and possibly a reaction from the insulin pump may occur, but this is not considered in more detail here. What is relevant within the context of the present invention is that the insulin pump 1 now, as one alternative, forwards each message 12 from the blood sugar meter 10 to the communication appliance 3 via the short range radio link 2. The communication appliance forwards it to the appliance 8, this applying to every message or just to selected messages, as has already been explained. Messages from the blood sugar meter 10 are therefore forwarded to the third party via the infusion pump 1.

In another embodiment, which is indicated by the arrow 13, the blood sugar meter 10 communicates directly with the communication appliance 3.

It should be under stood that the present invention and its components, e.g., an infusion pump, blood sugar meter, telephones, processor(s), sensor(s), etc., may comprise any suitable electrical components and circuitry, chips, boards, communication devices, microprocessors, inputs, outputs, displays, control components, software, hardware, etc.

In some preferred embodiments, the wireless connection, e.g., using the Bluetooth standard, between the medical appliance 1 or 10, e.g., an insulin pump or blood sugar meter or a continuously measuring sensor, and a communication appliance 3, e.g., a Smartphone, which for its part is connected to a radio network 6, therefore allows the remote monitoring to be carried out easily. An advantage is that a wireless, fully automated system is obtained or provided which does not require any operator control by the wearer of the infusion pump or the user of the blood sugar meter. In other words, the messages and alarms are sent and forwarded via the radio network automatically without requiring any operator control. A preferred form of the communication appliance 3 as a telephone or Smartphone provides the advantage that no additional appliances need be worn by the patient. In some embodiments, remote monitoring in accordance with the present invention allows a monitoring person to establish, e.g., whether measurements are being taken regularly and whether the pump functions are being carried out properly. This is useful particularly when monitoring children who are wearing an infusion pump.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A medical device adapted to transmit messages via a communication network to a communication device, the communication device adapted to send the same or another message via the same or another communication network to a further communication device.

2. The medical device according to claim 1, wherein the medical device is one of an insulin pump, a blood sugar meter or a continuously or quasi-continuously operating sensor.

3. The medical device according to claim 2, wherein the communication device is one of a telephone or a pager.

4. The medical device according to claim 3, wherein message sending is automatic and is initiated by the medical device.

5. A method comprising the steps of producing a message in a medical device, transmitting the message to a communication device and sending the same or another message to another communication device, whereby the medical device may be remotely monitored.

6. The method according to claim 5, wherein message sending is automatic and is initiated by the medical device.

7. The medical device according to claim 6, wherein the medical device is one of an insulin pump, a blood sugar meter or a continuously or quasi-continuously operating sensor.

8. A method for monitoring a medical appliance, comprising the following steps:
producing a message in the medical appliance,
transmitting the message wirelessly via a short range radio link to a communication appliance which is designed for wireless connection setup to a telephone or radio paging network, and
forwarding the message via the telephone or radio paging network to a further wireless communication appliance connected to the same telephone or radio paging network.

9. The method according to claim 8, wherein the medical appliance is an insulin pump and produces messages on the basis of at least one of a pump alarm, a bolus release or another pump event.

10. The method according to claim 8, wherein the medical appliance is a blood sugar meter and produces messages on the basis of at least one of blood sugar measured values or a meter event.

11. The method according to claim 8, wherein the medical appliance is a continuously or quasi-continuously operating sensor.

12. The method according to claim 9, wherein the insulin pump produces a message on the basis of a blood sugar measured value which has been sent to the insulin pump previously.

13. The method according to claim 12, wherein the measured value was sent via a short range radio link from a blood sugar meter to the insulin pump.

14. The method according to claim 8, wherein the first communication appliance is a wireless telephone or a pager.

15. The method according to claim 14, wherein the wireless telephone has the properties of a computer.

16. The method according to claim 8, wherein the telephone network is a GSM network, a UMTS network or another network and the radio paging network is a POCSAG network or another network.

17. The method according to claim 8, wherein the message is transmitted on the telephone network as an SMS message, an MMS message or using other transmission protocols or services.

18. The method according to claim 8, wherein the further communication appliance is one of a wireless telephone or a pager.

19. The method according to claim 8, wherein the further communication appliance is a wireless telephone.

20. The method according to claim 8, wherein the flow of messages occurs automatically and is initiated by the medical appliance.

21. The method according to claim 20, wherein in the communication appliance is adapted to assess whether or not the message needs to be forwarded to the further communication appliance.

22. An arrangement for transmitting a diabetes-related message from a diabetic to a remote person, comprising an insulin pump adapted to send a pump-related message via a short range radio network and one of a wireless telephone or pager adapted to receive the pump-related message via the short range radio network and, upon resultant automatic initiation, to send an SMS message or an MMS message reflecting the pump-related message to one of a further telephone or pager.

23. An arrangement for transmitting a diabetes-related message from a diabetic to a remote person, comprising a blood sugar meter adapted to send a meter-related message via a short range radio network and one of a wireless telephone or pager adapted to receive the meter-related message via the short range radio network and, upon resultant automatic initiation, to send an SMS message or an MMS message reflecting the meter-related message to one of a further telephone or pager.

24. An arrangement for transmitting a diabetes-related message from a diabetic to a remote person, comprising an insulin pump adapted to receive a message via a short range radio network, a blood sugar meter adapted to send a message via a short range radio network to the insulin pump, the insulin pump also being adapted to forward the message via a short range radio network, and a wireless telephone or a pager adapted to receive a message from the insulin pump via the short range radio network and, under resultant automatic initiation, to send an SMS message or MMS message containing the message from the insulin pump to a further telephone or a pager.

25. The arrangement according to claim 24, wherein the wireless telephone or the pager is adapted to check a message from the insulin pump or the blood sugar meter to determine whether or not it is a message which needs to be forwarded, whereupon the message is automatically forwarded or not forwarded.
